(19) United States
(12) Patent Application Publication
(10) Pub. No.: US 2024/0041359 A1
(43) Pub. Date: Feb. 8, 2024

(54) ANALYTE SENSOR

(71) Applicant: PercuSense, Inc., Valencia, CA (US)

(72) Inventor: KATHERINE WOLFE, DUNWOODY, GA (US)

(73) Assignee: PercuSense, Inc., VALENCIA, CA (US)

(21) Appl. No.: 18/230,596

(22) Filed: Aug. 4, 2023

Related U.S. Application Data
(60) Provisional application No. 63/395,174, filed on Aug. 4, 2022.

(51) Int. Cl. A61B 5/145

(52) U.S. Cl. CPC A61B 5/14546; A61B 5/14532

(57) ABSTRACT

In one embodiment, a sensor assembly is disclosed. The sensor assembly includes a first analyte sensor being formed on a first substrate and a second analyte sensor being formed on a second substrate. Wherein the first analyte sensor is coupled to the second analyte sensor within a coupling area defined by an overlap between the first analyte sensor and the second analyte sensor.

### RELATED APPLICATIONS

This application claims the benefit of U.S. provisional application Ser. No. 63/395,174, filed on Aug. 4, 2022. The application listed above is hereby incorporated by reference in its entirety for all purposes.

### FIELD OF THE INVENTION

The present invention is generally directed to devices and methods that perform in vivo monitoring of an analyte or analytes such as, but not limited glucose, lactate or ketones. In particular, the devices and methods are for electrochemical sensors that provide information regarding the presence or amount of an analyte or analytes within a subject.

### BACKGROUND OF THE INVENTION

Diabetes is a growing healthcare crisis, affecting nearly 30 million people in the United States. Approximately 10 percent of those affected require intensive glucose and insulin management. In hospital patients, hypoglycemia in both diabetic and non-diabetic patients is associated with increased cost and short- and long-term mortality.

Detecting and monitoring glucose levels within a subject can enable diagnosis of conditions associated with metabolism and general health. Detecting and monitoring analytes or molecules in addition to glucose can provide further insight regarding a subject's overall health. Commercially available real-time continuous glucose sensors are typically inserted percutaneously into interstitial fluid using sharps such as needles. Accordingly, using a single needle to insert a single sensor capable of measuring multiple analytes would be an improvement over multiple needles to insert multiple single analyte sensors. Moreover, there may be additional advantages gleaned by simplifying the assembly process by manufacturing different single analyte sensors separately and then joining the different sensors together into a single multianalyte sensor assembly.

However, the placement of such a multianalyte sensor assembly percutaneously may present some challenges. For example, because the multianalyte sensor assembly may be relatively thicker than the individual sensor, it may be beneficial to enable each individual sensor to flex independently of the other individual sensor. The ability to independently flex can improve both sensor longevity and general comfort of the subject.

Accordingly, it would be highly advantageous to enable simplified manufacturing processes for real-time, in-vivo, multianalyte sensors with improved flexibility to enhance patient comfort and sensor performance.

### BRIEF SUMMARY OF THE INVENTION

In one embodiment, a sensor assembly is disclosed. The sensor assembly includes a first analyte sensor being formed on a first substrate and a second analyte sensor being formed on a second substrate. Wherein the first analyte sensor is coupled to the second analyte sensor within a coupling area defined by an overlap between the first analyte sensor and the second analyte sensor.

In another embodiment, a method to fabricate a plurality of two-sided sensors where a portion of the first side of the sensor flexes independently of a second side of the sensor is disclosed. The method includes an operation to produce a plurality of first analyte sensors on a first substrate. The method further includes an operation to produce a plurality of second analyte sensors on a second substrate. Another operation is to produce a midlayer that includes coupling material and at least one air gap column. Still another operation is to couple the first substrate to the second substrate using the midlayer.

Other features and advantages of the invention will become apparent from the following detailed description, taken in conjunction with the accompanying drawings that illustrate, by way of example, various features of embodiments of the invention.

### BRIEF DESCRIPTION OF THE DRAWINGS

FIGS. 1A and 1B are exemplary illustrations of a top view of individual sensors, in accordance with embodiments of the present invention.

FIG. 1C is an exemplary illustration of a combined sensor that includes individual sensors, in accordance with embodiments of the present invention.

FIG. 2A is an exemplary illustration of a sensor, in accordance with embodiments of the present invention.

FIG. 2B is an exemplary illustration of the sensor after being folded along fold line, in accordance with embodiments of the present invention

FIG. 3A is an exemplary top view of a sensor in accordance with various embodiments of the present invention.

FIGS. 3B-3E are exemplary cross-section views of the sensor at various positions, in accordance with embodiments of the present invention.

FIG. 3F is an exemplary illustration showing the sensor in a first position without a force being applied and a second position with a force being applied, in accordance with embodiments of the present invention.

FIGS. 4A-4C are exemplary illustrations of various steps of fabrication for mass producing at least two types of sensors having different air gaps, in accordance with embodiments of the present invention.

FIG. 5A is an exemplary top view of a sensor, in accordance with embodiments of the present invention.

FIG. 5B is an exemplary illustration of a first sensor substrate and second sensor substrate, in accordance with embodiments of the present invention.

FIG. 5C is an exemplary illustration of a cross section first sensor substrate and second sensor substrate, at location D-D in FIG. 5A, in accordance with embodiments of the present invention.

FIG. 5D is an exemplary illustration of a cross-section of the sensor at location D-D after the first and second sensor substrates are coupled together, in accordance with embodiments of the present invention.

FIG. 6A is an exemplary top view of a sensor, in accordance with embodiments of the present invention.

FIG. 6B is an exemplary illustration of a cross section of a partial build up of a sensor at location D-D in FIG. 6A, in accordance with embodiments of the present invention.

FIG. 6C is an exemplary illustration of a cross-section of the sensor at location D-D after additional electrical insulators are laminated over the structure illustrated in FIG. 6B, in accordance with embodiments of the present invention.

### DETAILED DESCRIPTION

Presented below are embodiments of sensor configuration that is intended to enable continuous real-time in-vivo electrochemical sensing of an analyte or molecule of interest within a subject. The in-vivo measurement within a subject is typically performed in tissue such as, but not limited to subcutaneous tissue. However, various embodiments can be inserted into the vasculature, musculature or organ tissue. The sensor may include a working electrode along with a counter electrode and a reference electrode. Alternatively, many embodiments utilize a working electrode in conjunction with a combined counter/reference electrode.

Embodiments of the sensor can be configured to measure analytes such as lactate, ketones, glucose and the like. Furthermore, while some embodiments may be configured to measure a single or individual analyte, other embodiments can be configured to measure multiple analytes including various combinations of at least two or more molecules of interest such as lactate, ketone, glucose, oxygen, reactive oxygen and the like. In still other embodiments, the sensors may be configured with infusion sets to enable sensing of a single or multiple molecule of interest while also enabling delivery of an infusate from a single point of entry.

In many embodiments the sensors are combinations of individual sensors that are coupled together. For example, a glucose sensor can be coupled to a lactate sensor to create a single sensor capable of measuring or detecting both glucose and lactate. In many embodiments, the individual sensors are mechanically coupled together using physical features such as, but not limited to notches or cuts. In still other embodiments, the individual sensors are mechanically coupled together using coupling material such as, but not limited to adhesives. The embodiments described below each have at least four electrical conductors where two electrical conductors may be used as working electrodes and the remaining two electrical conductors are used as counter/reference electrodes. The embodiments disclosed below should not be construed as limiting as various combinations of one or more of each working electrode, counter electrode, reference electrode, and counter/reference electrode may be utilized to measure a single or multiple analytes.

As disclosed below, each individual sensor that is integrated into the combined sensor may have a laminate structure. Because the combined sensor is intended to be implanted within a subject, it may be beneficial to enable each individual sensor to flex and bend. By allowing each individual sensor to flex and bend independently, overall stress imparted to the laminate structure may be reduced and minimize the likelihood of delamination. Additional embodiments are disclosed below that locate the electrical conductors within the individual sensors as close as possible to the neutral plane to further remove a potential source of delamination.

For simplification and clarity, throughout this disclosure, the illustrations of cross-sections of sensors and sensor assemblies are of the sensor substrates before application of reactive chemistries, and additional layers that functionalize the sensor. The objective of this disclosure is to demonstrate methods and techniques that enable two-sided sensors to be built or constructed from single-sided sensors. The embodiments discussed below are intended to be exemplary and should not be viewed or construed as discrete individual embodiments. Rather, where possible, individual features discussed in each embodiment should be considered transferable to the other embodiments.

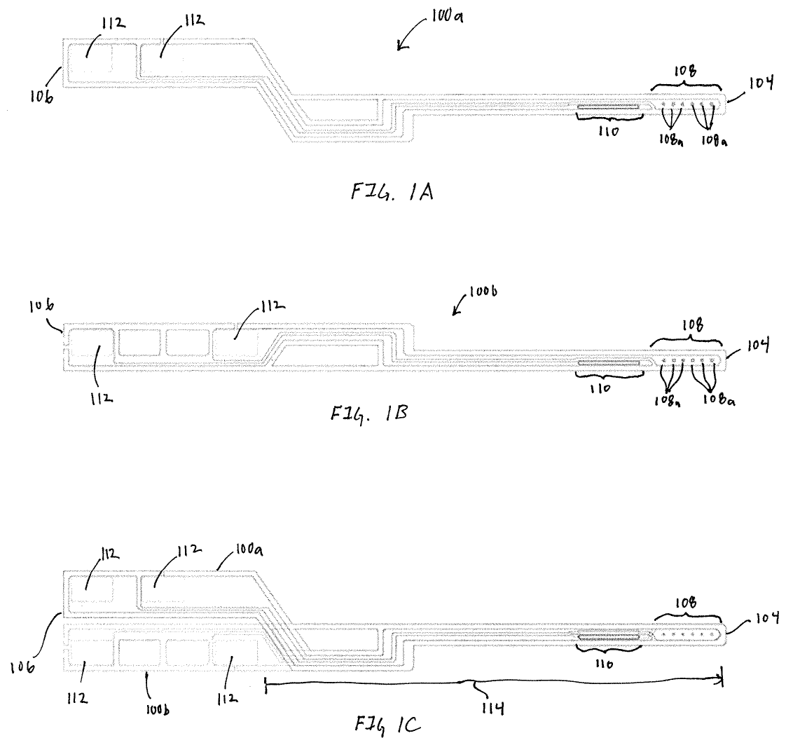

FIGS. 1A and 1B are exemplary illustrations of a top view of individual sensors 100a and 100b, in accordance with embodiments of the present invention. In many embodiments the sensor 100a and 100b can be operated independently. For example, in some embodiments sensor 100a may be configured to measure a first analyte or molecule of interest while sensor 100b is configured to measure a second analyte or molecule of interest. Exemplary, non-limiting analytes or molecules of interest include, but are not limited to glucose, lactate, ketone, tissue-oxygen, or other analytes capable of being detected or measured using electrochemistry. While it may be possible for the sensor 100a and sensor 100b to measure different analytes, in some embodiments the sensors 100a and 100b may be configured to measure or detect the same analyte.

Because each sensor 100a and 100b is capable of being operated independently, there are many commonly shared elements. For example, commonly shared elements on each sensor 100a and 100b include a distal end 104 that is intended to be inserted into a subject. Located toward the distal end 104 are a working electrode 108 and a combined counter/reference electrode 110. As illustrated, the working electrode 108 is composed of multiple openings 108a in an insulation layer that exposes an array of openings on an electrical conductor that form an array. This should not be construed as limiting as other embodiments of the working electrode 108 can be a single opening. Similarly, the illustration of the counter/reference electrode 110 should not be construed as limiting. In alternative embodiments rather than single opening the counter/reference electrode may be an array of openings similar to the working electrode.

Moreover, the relative size and area ratio between the working electrode 108 and counter/reference electrode 110 should not be construed as limiting. In various embodiments, depending on the analyte being detected or measured via the working electrode, it may be beneficial to use larger or smaller openings relative to the counter/reference electrode 110. Also shared among sensor 100a and 100b is the proximal end 106 that is not intended to be inserted into the subject. Located toward the proximal end 106 are contact pads 112 that enable electrical contact between electronics components capable of powering and detecting electrochemical signals generated between the working electrode 108 and the counter/reference electrode 110. Electrical traces connect the respective electrode to the corresponding contact pad 112.

Furthermore, the use of the combined counter/reference electrode should not be construed as limiting. In alternative embodiments rather than a two electrode system (e.g., working electrode and combined counter/reference electrode), a three electrode system having a working electrode, counter electrode, and reference electrode may be used. One skilled in the art should recognize that the use of the three electrode system can be implemented with the inclusion of a third electrode and associated electrical trace that electrically connects the third electrode to another contact pad. In embodiments utilizing a three electrode system it may be required to slightly increase the physical dimensions of the sensor 100a or sensor 100b to accommodate the third electrode near the proximal end 104.

In preferred embodiments the sensor 100a and 100b are made using flexible materials.

For example, in many embodiments the working electrode 108 and counter/reference electrode 110 can be built over a substrate of thin, flexible electrical conductor such as, but not limited to stainless steel. Exemplary grades of stainless steel include, but are not limited to grade 316 or grade 304 stainless steel. Additionally, in addition to the type or grade of stainless steel, the electrical conductor can be selected from a range of different thicknesses. For example, in some embodiments the thickness of the electrical conductor may be between about 0.00025 inches and 0.005 inches. In many embodiments, the thickness of the electrical conductor may be between about 0.0005 inches and 0.003 inches. The exemplary materials and thicknesses discussed above should not be construed as limiting. In other embodiments, the electrical conductor can be alternative flexible materials such as, but not limited to alloys that include gold, silver, platinum or even flexible materials coated with electrical conductors such as, but not limited to carbon nanotubes or other flexible electrical conductors.

In many embodiments the substrate or electrical conductor is laminated or sandwiched between electrical insulators such as, but not limited to polyamide and other flexible, electrically non-conductive materials. Accordingly, the electrical insulators completely cover, mask, or envelope the electrical conductor. Openings in at least one of the insulation layers enables formation of the contact pads 112 along with the openings to the substrate that form the working electrode 108 and counter/reference electrode 110. As the contact pads 112 are formed by removing electrical insulation from the substrate, the contact pads 112 can be located on a same side as the working electrode 108 and counter/reference electrode 110. In other embodiments, the contact pads 112 can be located on a side opposite the working electrode 108 and counter/reference electrode 110. The advantages of being able to form the contact pads 112 on either side of the sensor 100a or 100b will be discussed in more detail below regarding FIG. 1C.

FIG. 1C is an exemplary illustration of a combined sensor 102 that includes sensors 100a and 100b, in accordance with embodiments of the present invention. In some embodiments, the sensor 102 is formed by pairing the sensor 100a to the sensor 100b. In many embodiments the pairing of sensor 100a and sensor 100b is achieved using physical features such as, but not limited to notches or cuts within, or formed upon each sensor 100a and 100b. In these embodiments, complimentary notches pair the sensors 100a and 100b together. In other embodiments, the sensors 100a and 100b are paired together via static adhesion. By pairing the sensors 100a and 100b each sensor 100a or 100b can flex or bend somewhat independent of the other sensor. This enables each sensor 100a and 100b to be compliant with movement of a subject wearing the sensor.

While in some embodiments, it may be desirable to have each sensor 100a and 100b move independently, in other embodiments the sensor 102 is formed by coupling sensor 100a to sensor 100b within a coupling area 114. The coupling area 114 is defined as portions of each sensor 100a and 100b that overlap. In many embodiments, the sensor 100a is coupled to the sensor 100b using adhesives such as, but not limited to acrylic, epoxy, or combinations thereof. In still other embodiments, other coupling techniques or methods can be used to secure or affix the coupling area 114 of sensors 100a and 100b. Note that the coupling area 114 is defined as the entirety of the overlapping portions of sensor 100a and sensor 100b. However, it should be noted that in some embodiments select areas of the overlapping portions of the sensors 100a and 100b are coupled together. Thus, in some embodiments, the entirety of the coupling area 114 may be coupled while in other embodiments, only select areas or portions of the coupling area 114 are affixed together. In embodiments where the sensor 102 is formed by coupling select areas within the coupling area 114, each sensor 100a and 100b can retain some ability to flex independently of the other sensor.

Sensor 102 is two-sided meaning that sensor 100a is located on the top while sensor 100b is located on the bottom. In preferred embodiments, sensor 100b is “flipped over” so the working electrode 108 and counter/reference electrode 110 of sensor 100b are not covered or obscured by the sensor 100a. In FIG. 1C, sensor 100a is in the same orientation as illustrated in FIG. 1A, i.e., a top view. However, sensor 100b is “flipped over”, or mirrored across a horizontal line, so the top side illustrated in FIG. 1B is on the backside of the sensor 102.

The ability to place the contact pads 112 on either side of the sensors 100a and 100b enables various embodiments where the contacts pads 112 for sensor 102 are located on either the same side (top or bottom), or on both sides (top and bottom). It may be advantageous to form contact pads on a single side or both sides depending on the configuration of the electronics being connected to the sensor 102.

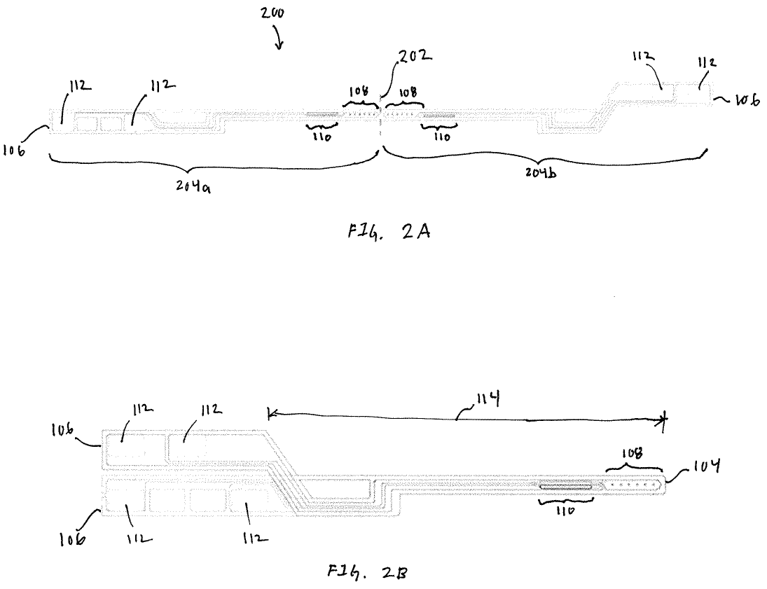

FIG. 2A is an exemplary illustration of sensor 200, in accordance with embodiments of the present invention. Sensor 200 is intended to be a two-sided sensor that is folded along a fold line 202. Sensor 200 includes first half 204a and second half 204b where the first half 204a and the second half 204b further includes elements such as the proximal end 106, contact pads 112, counter/reference electrodes 110 and working electrodes 108.

FIG. 2B is an exemplary illustration of the sensor 200 after being folded along fold line 202, in accordance with embodiments of the present invention. Accordingly, fold line 202 has become distal end 104. In some embodiments, after folding along fold line 202, the first half 204a and the second half 204b remain connected along fold line 202 and each half 204 and 204b is allowed to flex or move independently from the other half. However, in other embodiments, FIG. 2B further includes the coupling area 114 where portions of the first half 204a and the second half 204b can be coupled together using similar techniques to those discussed above. This enables either the entire coupling area 114 or select areas or portions within the coupling area 114 to be coupled together. In embodiments where only select areas within the coupling area 114 are coupled together, the remaining portions of the first half 204a and the second half 204b retain the ability to flex independently of the other half.

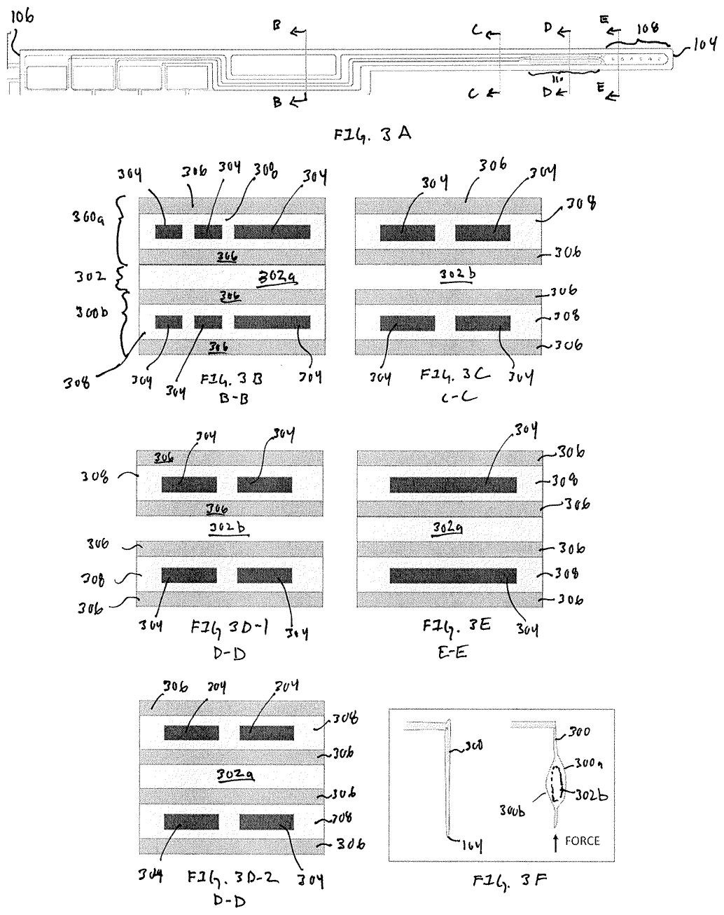

FIG. 3A is an exemplary top view of a sensor 300 in accordance with various embodiments of the present invention. The sensor 300 is a two-sided sensor that includes a first sensor 300a and a second sensor 300b. The sensor 300 is intended to illustrate locations for cross-sections that illustrate how an air gap can be selectively positioned between the first sensor 300a and the second sensor 300b to improve overall flexibility of the sensor 300.

FIGS. 3B-3E are exemplary cross-section views of the sensor 300 at various positions, in accordance with embodiments of the present invention. Across FIGS. 3B-3E, various cross-sections of the first sensor 300a and the second sensor 300b are shown, along with midlayer 302. Across FIGS. 36-3E, midlayer 302 can include a coupling material 302a or an air gap 302b, also referred to as void. Consistent across FIGS. 3B-3E are electrical conductors 304, electrical insulator 306 and adhesive 308. As illustrated, the conductors 304 are typically directly in contact with adhesive 308. Moreover, the adhesive 308 couples the electrical insulator 306 to the conductors 304. In FIGS. 3B-3E the electrical conductors 304 within the first sensor 300a and the second sensor 300b are substantially the same thickness. However, in many embodiments, it may be preferable to have the electrical conductors 304 within the first sensor 300a to be a different thickness than the electrical conductors 304 in the second sensor 300b. Variations or difference between thicknesses of electrical conductors between the first sensor 300a and the second sensor 300b may be because the first sensor 300a may be configured to detect or sense a different analyte or molecule than the second sensor 300b.

FIG. 3B is an exemplary cross-section at location B-B on sensor 300. In FIG. 3B, the midlayer 302 between the first sensor 300a to the second sensor 300b may be a coupling material 302a such as, but not limited to adhesives such as acrylic, epoxy or combinations thereof. In other embodiments, different coupling techniques may be used to couple portions of the first sensor 300a to the second sensor 300b.

FIG. 3C is an exemplary cross-section at location C-C on sensor 300. In FIG. 3C, the midlayer 302 is an air gap 302b. The air gap 302b is a void that allows first sensor 300a and second sensor 300b to move or flex independently, or separately, from each other. While the first and second sensors 300a and 300b remain coupled together at location B-B, at location C-C there is a physical space or separation between the first and second sensors 300a and 300b.

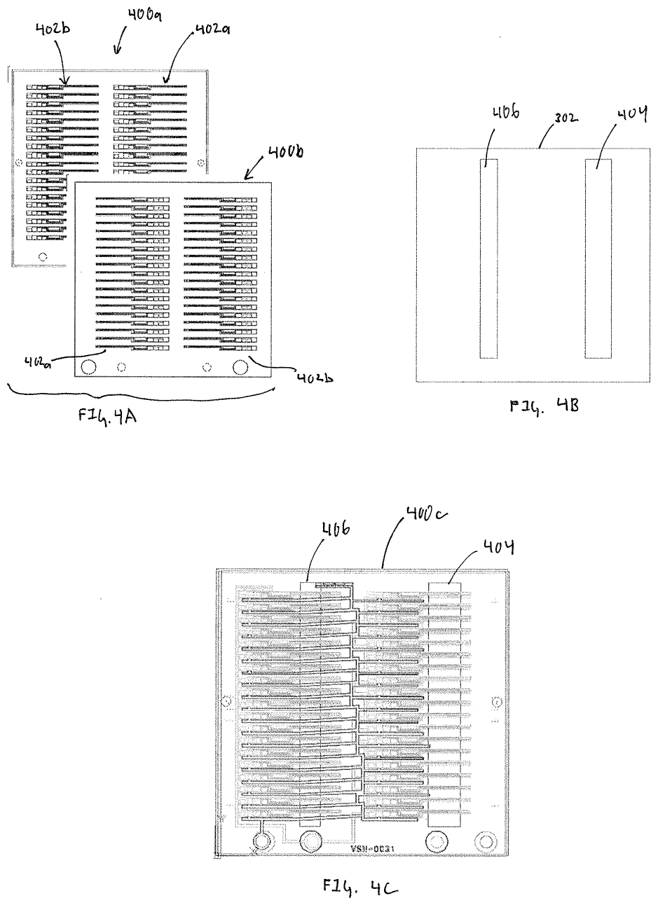

FIGS. 3D-1 and 3D-2 are exemplary cross-sections of two different embodiments of sensor 300 at location D-D. FIG. 3D-1 is identical to FIG. 3C because the midlayer 302 is an air gap 302b between the first sensor and the second sensor 300a and 300b. However, in FIG. 3D-2, the midlayer 302 includes a coupling material 302a. As will be discussed below regarding FIGS. 4A-4C, sensors 300 having different sized air gap 302a can be mass produced at the same time thereby enabling optimization or customization of sensor flexibility based on an intended purpose.

FIG. 3E is an exemplary cross-section at location E-E, through the working electrode 108 located at the distal end 104. At location E-E, the midlayer 302 again includes a coupling material 302a. In many embodiments it may be beneficial to couple the first sensor and second sensor 300a and 300b at the distal end 104 to enable insertion of the sensor through the skin of a subject.

FIG. 3F is an exemplary illustration showing the sensor 300 in a first position without a force being applied and a second position with a force being applied, in accordance with embodiments of the present invention. Note that the sensor 300 has portions that are selectively coupled together and other portions that include an air gap 302b. Accordingly, without the force being applied in the first position, the sensor 300, that includes sensor 300a and sensor 300b remain back-to-back. With the force being applied to the distal end 104, sensor 300a and sensor 300b are capable of flexing independently of each other at the air gap 302b.

FIGS. 4A-4C are exemplary illustrations of various steps of fabrication for mass producing at least two types of sensors having different air gaps, in accordance with embodiments of the present invention. FIG. 4A is an exemplary illustration of a first sensor substrate 400a and a second sensor substrate 400b. Each sensor substrate 400a and 400b includes multiple sensors similar to those illustrated in FIG. 3A. However, in FIG. 4A, the individual sensors that are shown in FIG. 3A have not been singulated or separated and are on one contiguous sheet. Both the first sensor substrate 400a and 400b have a first column of sensors 402a and a second column of sensors 402b. Note that in preferred embodiments, sensor substrates 400a and 400b would be fully processed and functionalized so singulation of individual sensors from either substrate 400a or substrate 400b would result in a functional single-sided sensor.

FIG. 4B is an exemplary illustration of midlayer 302 that is used to couple the first sensor substrate 400a to the second sensor substrate 400b. As previously discussed, in many embodiments the midlayer 302 includes the coupling material such as an adhesive. In preferred embodiments, the midlayer 302 is applied between the first sensor substrate 400a and the second sensor substrate 400b. As illustrated, the midlayer 302 includes a first column air gap 404 and a second column air gap 406. Both the first and second column air gaps 404 and 406 are opening in the midlayer 302 that are devoid of the coupling material. Accordingly, in FIG. 4B, the first column air gap 404 corresponds to the cross-section in FIG. 3D-1 while the second column air gap 406 corresponds to the cross-section in FIG. 3D-2.

FIG. 4C is an exemplary illustration of sensor sheet 400c that results from coupling the first sensor substrate 400a and the second sensor substrate 400b together using the midlayer 302. Still visible in the sensor sheet 400c are the first column air gap 404 and the second column air gap 406 under the respective first column of sensors 402a and the second column of sensors 402b. While the embodiment includes illustrations of at least two different air gaps 404 and 406, in other embodiments additional or fewer air gaps can be implemented by changing any of the dimensions of the first and second column air gaps 404 and 406. This can enable sensors having various or different flex characteristics to be fabricated simultaneously.

After the sensor sheet 400c is created by coupling the first sensor substrate 400a to the second sensor substrate 400b using midlayer 302, the individual sensors can be singulated or separated from the sensor sheet 400c using a variety of techniques. In some embodiments mechanical techniques such as shearing or cutting is used to separate the individual sensors from the sensor sheet 400c. In other embodiments, laser ablation can be used to detach the individual sensor from the sensor sheet 400c. In still other embodiments, combinations of techniques may be used such as partially separating a portion using laser ablation and shearing or cutting for the remainder of the individual sensor. The specific techniques described above should not be construed as limiting as other methods to singulate the sensors from the sensor sheet 400c may be used.

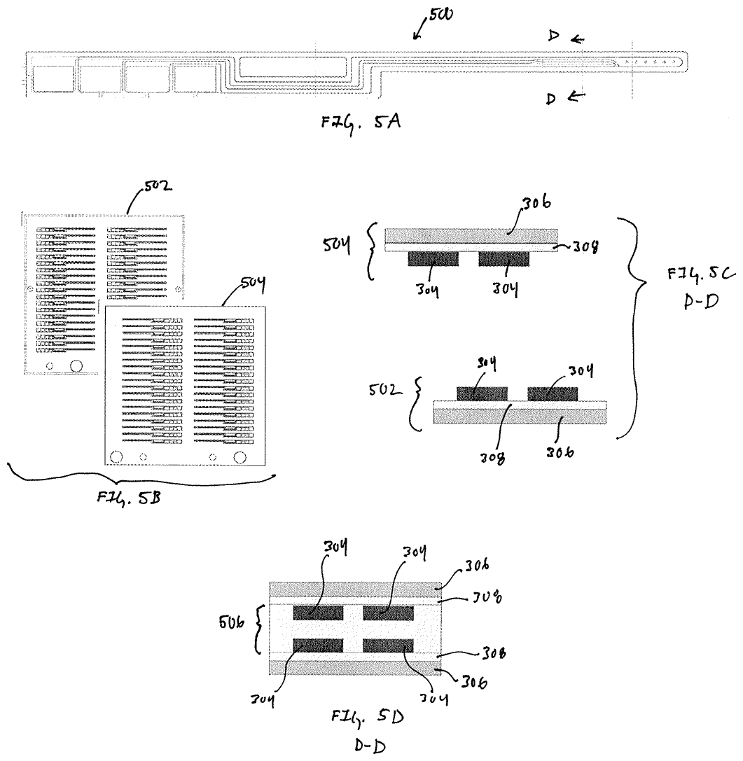

FIG. 5A is an exemplary top view of a sensor 500, in accordance with embodiments of the present invention. The sensor 500 is a two-sided sensor that includes a first sensor and a second sensor. The sensor 500 is intended to illustrate a location for a cross-section that illustrates a technique to create a two-sided sensor having a reduced thickness relative to embodiments discussed above.

FIG. 5B is an exemplary illustration of a first sensor substrate 502 and second sensor substrate 504, in accordance with embodiments of the present invention. In many embodiments, the first sensor substrate 502 is coupled to the second sensor substrate 504 to form a sensor sheet of two-sided sensors arranged in two columns.

FIG. 5C is an exemplary illustration of a cross section first sensor substrate 502 and second sensor substrate 504, at location D-D in FIG. 5A, in accordance with embodiments of the present invention. Both the first sensor substrate 502 and the second sensor substrate 504 include conductor 304, electrical insulator 306 and adhesive 308. In preferred embodiments, the adhesive 308 is laminated to the electrical insulator 306. The conductor 304 is coupled to the adhesive 308 and patterned or etched to form the electrical traces, contact pads and areas for the working electrode and counter/reference electrode.

FIG. 5D is an exemplary illustration of a cross-section of the sensor 500 at location D-D after the first and second sensor substrates 502 and 504 are coupled together, in accordance with embodiments of the present invention. As illustrated in FIG. 5D, midlayer 506 couples the first and second sensor substrates 502 and 504 together. This brings the conductors 304 closer to a neutral plane within the sensor 500. Moreover, in many embodiments, the midlayer 506 is a coupling material such as, but not limited to adhesives. While the midlayer 302 in FIG. 3D-2 is adhesive and the midlayer 506 in FIG. 5D is adhesive, note that in FIG. 5D two layers of electrical insulator 306 have been eliminated relative to FIG. 3D-2. Accordingly, the reduction in materials can be beneficial as the conductors are closer to the neutral plan.

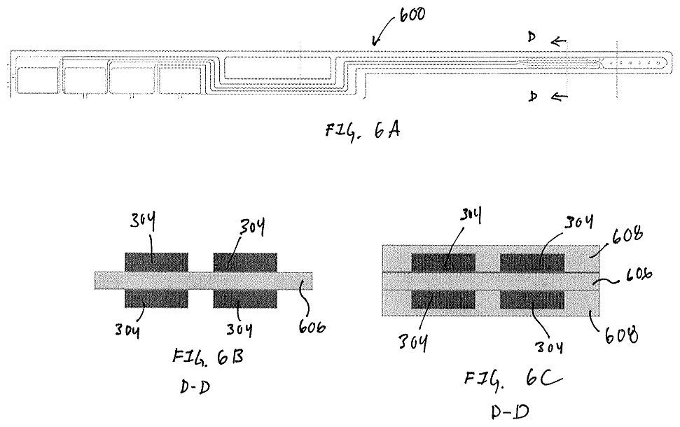

FIG. 6A is an exemplary top view of a sensor 600, in accordance with embodiments of the present invention. The sensor 600 is a two-sided sensor that includes at least four electrical traces to enable at least one working electrode and at least one counter/reference electrode. The sensor 600 is intended to illustrate a location for a cross-section that illustrates a technique to create a two-sided sensor having a reduced thickness relative to embodiments discussed above.

FIG. 6B is an exemplary illustration of a cross section of a partial build up of a sensor 600 at location D-D in FIG. 6A, in accordance with embodiments of the present invention. In FIG. 6B the electrical conductor 304 is laminated to an electrical insulator 606. Subsequent operations remove unwanted portions of the electrical conductor 304 to form the traces 304 associated with the contact pads, working electrodes and counter/reference electrodes. Note that by laminating the electrical conductor 304 directly to the electrical insulator 606, the electrical conductor 304 is located closer to the neutral plane. Additionally, the electrical insulator 606 becomes the equivalent of the midlayer discussed above. One benefit of locating the electrical conductor 304 closer to the neutral plane is that it can help minimize a source or location of delamination of the sensor.

FIG. 6C is an exemplary illustration of a cross-section of the sensor 600 at location D-D after additional electrical insulators 608 are laminated over the structure illustrated in FIG. 6B, in accordance with embodiments of the present invention. As illustrated in FIG. 6C, electrical insulator 606 couples or retains the traces formed from the electrical conductor 304. This brings the conductor 304 closer to a neutral plane within the sensor 600. Additionally, the reduction in materials further results in a sensor with a reduced thickness and potential cost savings associated with less materials.

In some embodiments single or multiple air gaps are incorporated into a two-sided sensor to improve or enhance flexibility of the sensor assembly. In still other embodiments, the electrical conductors are located closer to the neutral plane to improve sensor assembly flexibility. An additional benefit of shifting or moving the electrical conductors closer to the neutral plan is the reduction of the likelihood of delamination or subsequent materials or layers placed over the substrate. For simplification and clarity, throughout this disclosure, the illustrations of cross-sections of sensors and sensor assemblies are of the sensor substrates before applications of reactive chemistries, and additional layers that functionalize the sensor.

The objective of this disclosure is to demonstrate methods and techniques that enable two-sided sensors to be built or constructed from single-sided sensors.

In many embodiments, additional features or elements can be included, added or substituted for some or all of the exemplary features described above. Alternatively, in other embodiments, fewer features or elements can be included or removed from the exemplary features described above. In still other embodiments, where possible, combinations of elements or features discussed or disclosed incongruously may be combined together in a single embodiment rather than discreetly or in the specific combinations described in the exemplary description found above. Accordingly, while the description above refers to particular embodiments of the invention, it will be understood that many modifications or combinations of the disclosed embodiments may be made without departing from the spirit thereof. The presently disclosed embodiments are therefore to be considered in all respects as illustrative and not restrictive.

1. A sensor assembly comprising:
a first analyte sensor being formed on a first substrate;
a second analyte sensor being formed on a second substrate;
wherein the first analyte sensor is coupled to the second analyte sensor within a coupling area defined by an overlap between the first analyte sensor and the second analyte sensor.

2. The sensor assembly described in claim 1, wherein the first analyte sensor has a first side and a second side, and further includes a working electrode and counter/reference electrode formed on the first side of the first substrate.

3. The sensor assembly described in claim 2, wherein the second analyte sensor has a first side and a second side, and further includes a working electrode and counter/reference electrode formed on a first side of the second substrate.

4. The sensor assembly described in claim 3, wherein the second side of the first analyte sensor is coupled to the second side of the second analyte sensor.

5. The sensor assembly described in claim 4, wherein adhesive couples the first analyte sensor to the second analyte sensor within the coupling area.

6. The sensor assembly described in claim 5, wherein the adhesive is selectively applied within the coupling area to create at least one air gap within the coupling area.

7. The sensor assembly described in claim 6, wherein the at least one air gap enables the first analyte sensor to flex independently of the second analyte sensor.

8. The sensor assembly described in claim 7, wherein the first analyte sensor includes electrical conductors being sandwiched between electrical insulators.

9. The sensor assembly described in claim 8, wherein the second analyte sensor includes electrical conductors being sandwiched between electrical insulators.

10. The sensor assembly described in claim 1, wherein the air gap does not extend to a distal end of the sensor assembly.

11. The sensor assembly described in claim 10, wherein the air gap does not extend to the proximal end of the sensor assembly.

12. The sensor assembly described in claim 1, wherein the first substrate and the second substrate have different thicknesses.

13. The sensor assembly described in claim 1, wherein the first substrate and the second substrate are a same thickness.

14. A method to fabricate a plurality of two-sided sensors where a portion of a first side of the sensor flexes independently of a second side of the sensor, comprising:
producing a plurality of first analyte sensors on a first substrate;
producing a plurality of second analyte sensors on a second substrate;
producing a midlayer that includes coupling material and at least one air gap column;
coupling the first substrate to the second substrate using the midlayer.

15. The method of claim 14, wherein the plurality of first analyte sensors are produced on a first side of the first substrate.

16. The method of claim 15, wherein the plurality of the second analyte sensors are produced on a first side of the second substrate.

17. The method of claim 16, wherein the midlayer is coupled to a second side of the first substrate.

18. The method of claim 17, wherein the midlayer is coupled to a second side of the second substrate.

19. The method of claim 18, wherein the midlayer further includes a second air gap column.

20. The method of claim 19, wherein the first air gap column and the second air gap column have different widths.
